(19) United States
(12) Patent Application Publication
(10) Pub. No.: US 2021/0038508 A1
(43) Pub. Date: Feb. 11, 2021

(54) FOAM FORMULATIONS AND DELIVERY METHODS TO THE BODY

(71) Applicant: Slate Therapeutics, Inc., Berkeley, CA (US)

(72) Inventors: Gary Binyamin, Berkeley, CA (US); Guillermo Piva, San Ramon, CA (US); Eitan Konstantino, Orinda, CA (US); Tanhum Feld, Merhavya (IL)

(73) Assignee: Slate Therapeutics, Inc., Berkeley, CA (US)

(21) Appl. No.: 17/077,973

(22) Filed: Oct. 22, 2020

Related U.S. Application Data
(63) Continuation of application No. PCT/US2019/033173, filed on May 20, 2019.
(60) Provisional application No. 62/673,896, filed on May 19, 2018.

(51) Int. Cl. A61K 9/00; A61M 15/00; A61M 15/08; A61K 9/12; A61K 9/107; A61K 9/51; A61K 47/26; A61K 47/10

(52) U.S. Cl. CPC A61K 9/0043; A61M 15/009; A61M 15/08; A61M 15/002; A61K 47/10; A61K 9/107; A61K 9/5123; A61K 47/26; A61K 9/122

(57) ABSTRACT

A liquid drug delivery formulation forms a foam when combined with a gas source. The formulation includes water, at least one hydrophilic solvent, at least one surfactant or foaming agent, and a drug particle which is a mixture of a therapeutic agent and an excipient. The formulations are useful for delivering foams to body cavities and lumens, such as the sinuses for treating sinusitis and other conditions.

### CROSS-REFERENCE TO RELATED APPLICATION

This application is a continuation of PCT Application No. PCT/2019/033173 (Attorney Docket No. 56387-703.601), filed May 20, 2019, which claims the benefit of U.S. Provisional Application No. 62/673,896 (Attorney Docket No. 56387-703.101), filed on May 19, 2018, the full disclosures of which are incorporated herein by reference.

### BACKGROUND OF THE INVENTION

### 1. Field of the Invention

The present application relates generally to drug delivery and, more particularly, to the use of gas foam to fill and conform to a body cavity or lumen and to provide effective contact and transfer between a medicament carried by the foam and a surface of the cavity or lumen.

Various foam formulations have been used in mucoadhesive drug delivery systems for drug delivery to body cavities and lumens, including nasal, buccal, ocular, gastro, vaginal, and rectal drug delivery systems. Such systems, however, have most often employed gel foams which provide poor surface contact between the gel foam and the body cavity or lumen, resulting in a low efficiency drug uptake. For example, when used to deliver drugs to a patient's sinuses, gel foams have a very low uptake in the target tissues.

Drug delivery using foams has been popular over the past two decades, especially for topical delivery. Along with creams, ointments, and gels these foams have clinically demonstrated safety and efficacy across a range of indications using a variety of therapeutic formulations.

It is therefore an objective of the present application to provide improved foam formulations, particularly gas-filled foams, capable of providing efficient drug delivery to the sinuses and other body cavities and lumens.

### 2. Description of Background Art

Khan et al. J. Controlled Release, 2017; 268: 364-389, describe brain targeting drug delivery systems by the nasal route. Sonvico et al. Pharma. 2018; 10:34 describe surface-modified nano-carriers for nose-to-brain drug delivery. Arzhavitina and Streckel, Int. J. Pharma 2010; 394:1-17 describe the development of foam formulations for dermatological delivery. See also US2004/0151774; US2014/0243427; US2017/0079929; and EP1838381.

### SUMMARY OF THE INVENTION

The present invention provides foam formations suitable for delivering a wide variety of particulate medicaments to body cavities and lumens, such as nasal, buccal, ocular, gastro, vaginal, and rectal areas. In use, the foam fills a volume of a body cavity or lumen and conforms to its surface, resulting in a high contact surface area between the cavity or lumen and the medicament to achieve efficient drug delivery. The foam formulations herein are intended primarily for the treatment of body cavities or lumens, but could also find use in topical delivery of medicaments and other substances to other tissue surfaces. Delivery of a propagating foam increases the efficiency of the treatment and allows partial filling of sinus cavities and increased surface contact when compared to conventional treatment with nasal spray, aerosols, or nebulizers. The presence of an appropriate therapeutic in the foam allows for passive uptake due to the surface contact. Inclusion of therapeutic components as part of a formulation for drug delivery allows the foam to impart a local, drug-related effect.

The present invention comprises a foam-based liquid suspension of surface active materials including, for example emulsifiers and foaming agents, that are mixed or loaded with antibiotic, steroids or other therapeutic agents (which may include one or more excipients in the formulation) provides for an improved treatment method. The application of the foam may be combined or preceded with a washing agent to prepare the surface. For example, the nasal cavity may be rinsed or washed to remove debris or physiological fluids, allowing for the foam to be subsequently applied directly to the tissue.

The present invention is particularly suitable for delivery of drugs to the sinus of the head and nasal pharynx. The foams of the present invention are able to fill and occupy substantially an entire volume of the sinus and nasal cavities, including cavities of all shapes, including those that having irregular shapes and/or which are unique in other ways which it may be difficult to achieve a high surface area or good apposition using alternate local methods, e.g. treatment of the nasal mucosa of the sinuses. Sinuses include, but are not limited to the frontal sinus, sphenoid sinus, ethmoid sinuses, and maxillary sinus.

Sinusitis is the inflammation of one or more sinuses and presents with a variety of symptoms including fever, headaches, effected smell, coughing, etc. While systemic antibiotics are used in some cases, they are not common as a first-line treatment. The antibiotics of choice include agents that cover organisms causing acute sinusitis but also cover Staphylococcus species and anaerobes. These organisms include, but are not limited to S. pneumoniae, H influenzae, and M catarrhalis. Therapeutic options include amoxicillin-clavulanate, cefpodoxime proxetil, cefuroxime, gatifloxacin, moxifloxacin, and levofloxacin. In select cases, local corticosteroids have been used. Nasal irrigation, breathing high-humidity vapors, and gargling are demonstrated treatments of sinusitis and its symptoms. Locally delivered decongestant (e.g. oxymetazoline) or antihistamines can be used to address symptoms in the short-term, but are no used for long-term treatment. The foams of the present invention will be able to locally treat the sinuses with antibiotics, decongestants, antihistamines, and corticosteroids, individually or formulated together. Furthermore, the foam formulation could include components that enhance the surface, yielding similar results to irrigation or inhalation of vapors.

The present invention can also provide nose-to-brain drug delivery by delivery of medicaments to the olfactory mucosa residing in the superior turbinate of the nasal conchae. See the anatomy illustrated in FIGS. 1 and 2. Therapeutics delivered in the superior turbinate of the nasal conchae can directly impact the brain and by-pass the blood-brain-barrier that can inhibit systemic delivery methods. Most nasal delivery methods, including sprays, aerosols, and nebulizers, typically do not penetrate to the depth required for delivery in this region. The foams of the present invention provide a delivery system which is able to deliver a therapeutic (molecule, biologic, cell therapy) of interest in a desired dosage.

The foams of the present invention will comprise liquid colloidal foam including a dynamic dispersion of a gas phase in a continuous liquid medium. Each of the foams disclosed herein will include a hydrophilic liquid continuous phase containing a hydrophilic solvent and a foaming agent or surfactant in which a gas phase will be distributed. A soluble therapeutic can also be included in the foam or, alternatively, a dispersed phase will be included as a third phase (e.g., particles or micelles). The foams will also include at least one therapeutic ingredient and one excipient ingredient within an established ratio range.

Suitable hydrophilic solvents may include alcohols and polyalcohols, such as ethanol, propylene glycol, ethylene glycol, benzyl alcohol, and glycerol. Other suitable hydrophilic solvents include dimethylsulfoxide and dimethylformamide. Suitable hydrophilic solvents are typically present at a concentration in a range from 5% to 75% typically from 5% to 50%, by weight of the total liquid drug delivery formulation.

The foaming agent or surfactant is preferably an amphiphilic substance, having hydrophilic and hydrophobic segments, typically in a concentration range of 0.25-5% by weight of the total formulation. Ideally the maximum foaming ability is observed when the foaming agent or agents are at a concentration at or near the critical micelle concentration.¹ The surfactant in this system may additionally act on the surface of the target tissue to reduce/remove or penetrate barriers (e.g. lipid layers) to enhance, accelerate or otherwise affect the transfer of one or more therapeutics. ¹ critical micelle concentration (CMC) is defined as the concentration of surfactants above which micelles form and all additional surfactants added to the system go to micelles

The therapeutic agent or other medicament will have a beneficial biological activity, typically being a small molecule drug with a MW below 2000D, usually below 1000D. Illustrative examples include corticosteroids, antiproliferatives such as taxanes and -limus analogs, antibiotics and antibacterials, and other lipophilic anti-inflammatory derivatives) or biologics (e.g. antibodies, proteins, bacteria, cells).

The formulation will include at least one therapeutic agent or medicament but can also be a combination or mixture of more than one. For example, a small molecule therapeutic, in the form of an active pharmaceutical ingredient (API) should be included in the foam formulation at a concentration of <15%, preferably is in a range of 0.1%-5.0%. In the case of an emulsion, the concentration of the therapeutic or therapeutics will be defined by the limit of solubility in the solvent system.

The drug particles will include at least one excipient, optionally being a combination or mixture of two or more excipients. An excipient is an inactive ingredient intended to minimize API aggregation. Exemplary excipients include urea, iopromide, citrate esters, and lipophilic antioxidants. Examples of lipophilic antioxidants include but are not limited to, nordihydroguaiarectic acid, resveratrol, propyl gallate, butylated hydroxyl toluene, butylated hydroxyanisole, and ascorbate palitate. Use of lipophilic antioxidant excipients may be preferred in some instances to inhibit the agglomeration of particles including hydrophobic therapeutic agents (API) having limited water solubility. The weight ratio of drug-to-excipient in the particles is typically in a range from 3 to 1 to 0.5 to 1.

Preferred embodiments of the foam formulations will include a stabilizing agent. The stabilizing agent can have a concentration of 0.1%-10% and preferably around 1%. Exemplary stabilizing agents include but are not limited to polysaccharide and starch.

Preferred embodiments of the foam formulations will also include a thickening agent. The thickening agent can have a concentration of 0.1%-10% by weight and preferably around 1%. Suitable thickening agents include starch, xanthan, guar gum, locust bean gum, gum karaya, gum tragacanth, gum Arabic and cellulose derivatives.

Preferred embodiments of the foam formulations will include a gelling agent, typically in place of or in combination with a thickening agent. The gelling agent can have a concentration of 0.1%-10% by weight and preferably around 1%. Suitable gelling agents include hydrocolloids including alginate, pectin, carrageenan, gelatin, gellan and agar.

The combination of a surfactant-based carrier and a therapeutic agent will increase the biological effect or availability of the therapeutic agent, by disrupting mucus and lipids in the sinus cavities and thereby allowing direct access to the tissue (e.g., mucosal layer). Improving interaction between the therapeutic agents and the tissue through enhanced exposure of the sinus wall to the therapeutic agent and reducing/removing lipid barriers. In one embodiment, these are formulated together in a single foam formulation.

In particular, the at least one surfactant or foaming agent may be a material which disrupts mucus and/or lipids on a wall of the body cavity or lumen to enhance delivery of the drug particles to a mucosal layer on the wall of the body cavity or lumen. Such disruption of the mucus and/or lipids on a wall of the body cavity or lumen is particularly useful for the delivery of corticosteroids and other drugs to and through the mucosa of a nasal sinus.

In a second, alternative embodiment, a first surfactant foam formulation may contain only a surfactant for the purpose of clearing and preparing the mucosal surface. This may be a stand-alone treatment or can be followed by a second therapeutic treatment.

### BRIEF DESCRIPTION OF THE DRAWINGS

The foregoing and other objects, features and advantages of embodiments of the present inventive concepts will be apparent from the more particular description of preferred embodiments, as illustrated in the accompanying drawings in which like reference characters refer to the same or like elements. The drawings are not necessarily to scale, emphasis instead being placed upon illustrating the principles of the preferred embodiments.

FIGS. 1 and 2 illustrate the nasal anatomy relevant to the drug delivery method of the present invention.

FIGS. 3A and 3B show a transparent model of the nasal-sinus system representing a human anatomy. FIG. 3A shows an over-the-counter nasal spray, red color, deployed per the instructions for use through the nostril opening in the transparent. Results show a concentration in the nasal region. FIG. 3B shows the results of using a foam formulation of the present invention, red color, deployed through nostril opening in the same model

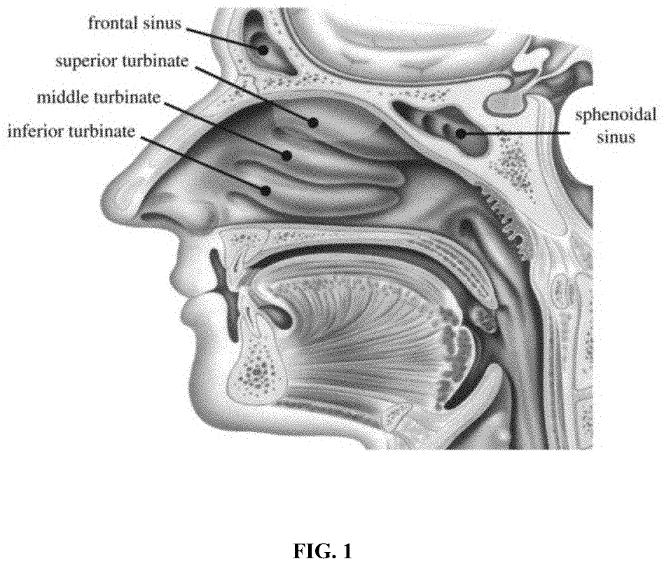

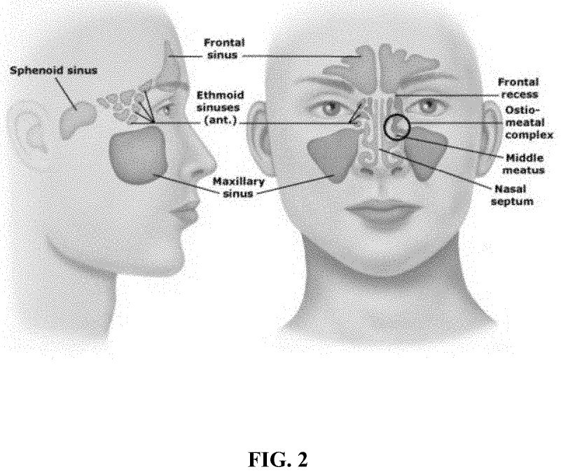

### DETAILED DESCRIPTION OF THE INVENTION

The present invention provides formation, application, and use of foam to fill the volume of a body cavity or lumen and conform to its surface, resulting in a high contact surface area between the cavity or lumen and material for the purpose of drug delivery. While particularly suited for nasal delivery of mucoadhesive drugs, the foam formulations and drug delivery methods of the present invention are also suitable for buccal, ocular, gastro, vaginal, and rectal drug delivery. The presence of an appropriate therapeutic in the foam formulation allows for passive uptake due at least primarily to the surface contact.

The active pharmaceutical ingredient is any therapy or treatment intended to act locally on the mucosa or for mucosal delivery with a targeted region of interested or for systemic delivery via a highly vascularized tissue bed. There is an established mechanism for transport to the brain, including via the olfactory and trigeminal nerves. Small molecules are a well-defined class of active pharmaceutical ingredients. Examples of small molecules intended to act locally on the mucosa are corticosteroids such as beclomethasone dipropionate, budesonide, flunisolide, fluticasone propionate, mometasone furoate, and triamcinolone acetonide. Examples of small molecules intended to act on the brain or CNS are fentanyl, zolmitriptan, nafarelin, and buserelin. Other classes of active pharmaceutical ingredients that can be delivered includes small molecules, macromolecules (e.g., biologic therapies such as live attenuated influenza vaccine), or cell-based therapies.

The active pharmaceutical agent should be in the form of microparticles, less than 10 nm and preferably less than 100 nm. The drug particles can be a single size or a distribution of sizes. For small molecule, active pharmaceutical ingredients microparticles may be comprised of as received powder or may be reformed using techniques known in the art, including precipitation and crystallization. Mechanical methods known to reduce particle size include grinding (wet or dry), milling, mortar & pestle, ultrasonic homogenization, electrohydraulic (arc cavitation) homogenization. Particles of appropriate size may be isolated by sieving or sorting techniques. Alternatively, the microparticles of the appropriate size can be comprised of a matrix (e.g., resorbable polymer, stabilizing scaffolding) containing the active pharmaceutical ingredient. The matrix might allow for a reservoir that elutes over time or for increased stability of the active pharmaceutical ingredient following delivery.

One example of generating recrystallized particles of a small molecule therapeutic having the appropriate size is by dispensing a dilute solution containing the active pharmaceutical ingredient using an ultrasonic spray to generate small droplets directly into an anti-solvent solution. Alternatively a nebulizer or Venturi-type system can be utilized that allows for the shower of a dilute solution containing the active pharmaceutical ingredient into an anti-solvent solution.

An aqueous foam is disclosed for the formulation of the active pharmaceutical ingredients. The foam should contain water, one or more active therapeutic ingredients, one or more surfactants (0.25-5%) and one or more excipients with a ratio between 0.5-1 to 3-1 of active therapeutic ingredient to excipient. One or more of the excipients should be a spacer molecule, used to avoided agglomeration of the active pharmaceutical ingredient. Additionally the formulation may include penetration enhancers, hydrophobic solvents, emulsifying agents, preservatives, dispersants, solubility enhancers, stabilizing agents, thickening agent, gelling agent, or other ingredients known in the art.

The foam formulation can be dispensed in a foam pump or squeeze foamer. The apparatus takes the liquid constituents of the formulation into the foam chamber and discharges it through a mesh as a foam. Air is the gas phase. The resultant foam has relatively large cells. Alternatively, a whipping siphon can be used to dispense the formulation. In this case carbon dioxide or nitrous oxide is dissolved in the liquid formulation at high pressure and then dispensed at atmospheric pressure. The change in pressure causes the gas to leave the solution with the resultant foam having relatively small cells.

A delivery catheter may be used to place the foam near to or at the area for delivery. A delivery catheter for sinus cavities includes a flexible elongated member with an atraumatic tip and tapered diameter and stiffness. Such catheters include a single lumen or more than one lumen. One or more lumens for dispensing and one or more additional lumens allowing for pressure outlet from sinus cavities during foam dispensing. One lumen could deliver the foam or foaming agents or liquid with therapeutic agent or other bio active material, while additional lumen or lumens provide air outlet and are connected to a free end or vacuum externally. During activation of the pump or a dispensing device liquid, foam or foaming agent will fill the sinus cavity and the additional lumen or lumens will provide air outlet (for example for pressure equalization) and by that enable filling of the blocked cavity or cavities that have single entry point.

Lumens for the transport of foam should be designed to minimize shear forces, which can degrade the foam quality. This can include reductions in friction between the foam and lumen surfaces which can be achieved by purposeful modifications to surface roughness, material choice, and or addition of coatings (e.g. hydrophilic or hydrophobic layers).

The additional lumen or lumens could be concentric or non-concentric to the materials dispensing lumen and could include multiple holes in its distal end to allow effective pressure equalization and air outlet from the sinus cavity back to the device or to any point out of the target cavity that is being treated.

The catheter system would be manually advanced to the location by the user. This could be achieved using a steerable distal assembly that is actuated from a distal handle. Alternatively, the catheter system could ride over a guidewire or through a sheath to reach the intended anatomy. Alternatively, the catheter could be designed to have sufficient distal-to-proximal force transmission and torqueability to allow for advancement.

### Method for Treatment Procedures

### Interventional Sinus Treatment

Sinuplasty or sinu-stenting procedures are performed under general anesthesia in the operating room, or in some circumstances in the office under local anesthesia. It does not require cutting or removal of bone or tissue, but does reshape the anatomy using mechanical or hydraulic forces; opening up blocked passages to facilitate drainage. Briefly, the procedure entails threading a guide wire through the nostril and into the target sinus cavity endoscopically through the nose. A balloon or stent then travels over the wire and is deployed, enlarging the sinus opening. Increased area of the opening allows for improved drainage. Saline lavage can then be used to flush the sinus of interest. Most patients go home the same day.

Sinuplasty techniques involving ballooning of a sinus cavity orifice is traumatic, breaking bones and cartilage, with the result being modification to the natural anatomy. Preferably permanent alteration of the anatomy should be avoided. The foam-based treatment provides more stable, long lasting and uniform drug delivery compared to liquid based drugs. This can potentially relieve inflammation and allow the cavity orifice to open naturally, without mechanical intervention.

The present invention provides a procedure in which a guide catheter is placed, and treatment occurs through it without mechanical effect to the anatomy. The procedure is comprised of irrigation and suction to wash off excessive liquids and mucus, and/or the delivery of foam to treat the sinus cavity. Treatment may include lavage, surface preparation, or therapeutic delivery. The space filling characteristics of the foam allow for intimate contact with all exposed surfaces and efficient treatment of the affected area. The guide catheter is used to direct the irrigation catheter and foam delivery catheter into the sinus cavity, and also for suction or exhaust.

The methods herein could be an alternative or addition to sinuplasty and sinu-stenting of the nasal cavity. Sinuplasty and/or sinu-stenting tools can provide improd accessibility to the sinus cavities for treatment.

### Rhinitis Treatment

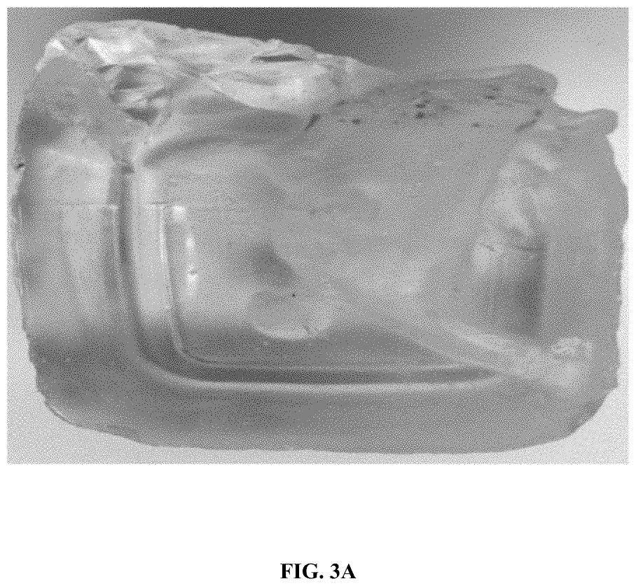

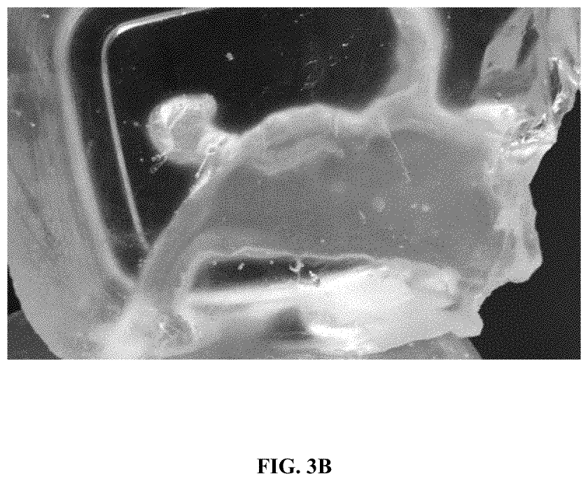

Treatment of non-allergic rhinitis is typically local delivery of over-the-counter therapies (e.g. saline, corticosteroids, antihistamines, decongestants, anti-cholinergics). Nasal sprays are one common delivery method, but may also include nasal irrigation (e.g neti-pot). It is well established that these treatments result in a minor amount being delivered to the nasal region, with up to 70% of the applied dose being swallowed and entering the gastrointestinal tract. The amount that is delivered within the nasal cavity is not well distributed and typically concentrated in the anterior nose. This has been confirmed using a model of the human nasal-sinus system, as shown in FIGS. 3A and 3B.

The current invention describes a space filling foam that can achieve contact with most sinus regions. Unfilled portions can be addressed if a route for gas escape can be established or the foam generation is initiated from within. A delivery catheter or multi-lumen delivery extension can be used to direct to areas of interest. The improved tissue contact minimizes delivery of the treatment to unintended regions and provides for improved deliver to the target tissue.

A direct nose-to-brain delivery route for therapies administered to the nasal cavity exists. The transport is described as occurring via the olfactory epithelium and/or the trigeminal nerves directly to the central nervous system (CNS), without passing through the blood brain barrier (BBB). This direct pathway to the CNS would be the preferred method for route of administration, especially for challenging therapies. Ensuring delivery and absorption within the appropriate nasal region, without excess loss to the gastrointestinal tract or due to the physiological/anatomical state of the nasal tissue are relevant considerations for this rout of administration.

The foam formulations are a targeted therapy that can be delivered to a specific location, with high surface coverage. Such a method would be a good delivery option for CNS therapies, including but not limited to small molecules, peptides, proteins, biologic molecules, vaccines, DNA, cells, polar molecules, nanoparticles, and vesicles.

### Foam Delivery System

Foam generation is dependent on the formulation going through a dispenser assembly which allows for the incorporation of a gas, for generation of the proper form. Certain formulations can be generated using non-aerosolized air as the gas component. This works by having a dual chamber pump, one for air and another for the formulation. Both come together in a mixing chamber and are forced through the same small nozzle, containing a mesh or screen.

Certain formulations can be generated by pressurizing with other gases, passing a pressurized liquid-gas through a valve to form an aerosol. Aerosolization gases suitable for human use include nitrous oxide or carbon dioxide. Other gases known in the art can also be utilized including but not limited to compressed gases (e.g. nitrogen, nitrous oxide carbon dioxide), hydrocarbons (e.g. butane, isobutene, propane), chlorofluorocarbons, hydrochlorofluorocarbons, dimethyl ether and hydrofluroalkanes (HFAs).

The present invention may also utilize a catheter system to deliver or transfer the foam to a certain cavity or body area is described. The foam can be generated at the proximal end and transferred through the catheter lumen or lumens to the delivery site. The foam formulation can be transferred through the catheter lumen or lumens and generated distally, prior to dispensing to the delivery site. The distal end that allows for delivery of the device to the intended anatomy and controls flow through the catheter resulting in foam discharge distally.

Alternatively, the foam can be delivered through a needle or other lumen, expanding to fill the volume and conform to the surface of the intended site of delivery, foaming at the time of the dispensing, distally at the exit point. The tip design can be optimized for aerosol delivery and selective release of therapeutic in the nostril, e.g. having an asymmetric nozzle design to achieve foam formation upon contact with back flow and space filling to achieve full coverage.

The foam will exit a lumen of the device. In some cases, space filling will allow for penetration of the foam through the established physiological system. In other cases, a lumen will need to be advanced to the cavity of interest prior to dispensing of the foam. Placement of the lumen can occur blindly, indirectly or directly. Examples of indirect placement may include use of a high-intensity light at the distal end of a wire or lumen to illuminate position in the nasal cavity during delivery. Direct delivery includes using visualization (camera, angiography, etc.).

Delivery catheters suitable for sinus cavities include a flexible elongated member with an atraumatic tip and tapered diameter and stiffness. Such catheters may include a single lumen or more than one lumen. One or more lumens for dispensing and one or more additional lumens allowing for pressure outlet from sinus cavities during foam dispensing. One lumen could deliver the foam or foaming agents or liquid with therapeutic agent or other bio active material, while additional lumen or lumens provide air outlet and are connected to a free end or vacuum externally. During activation of the pump or a dispensing device liquid, foam or foaming agent will fill the sinus cavity and the additional lumen or lumens will provide air outlet (for example for pressure equalization) and by that enable filling of the blocked cavity or cavities that have single entry point.

Foam delivery lumens should minimize shear forces which can degrade the foam quality. Forming or treating the luminal surfaces can reduce friction between the foam and lumen surfaces, eg. reducing luminal surface roughness, material choice, and/or addition of coatings (e.g. hydrophilic or hydrophobic layers).

The additional lumen or lumens could be concentric or non-concentric to the materials dispensing lumen and could include multiple holes in its distal end to allow effective pressure equalization and air outlet from the sinus cavity back to the device or to any point out of the target cavity that is being treated.

The catheter system would be manually advanced to the location by the user. This could be achieved using a steerable distal assembly that is actuated from a distal handle. Alternatively, the catheter system could ride over a guidewire or through a sheath to reach the intended anatomy. Alternatively, the catheter could be designed to have sufficient distal-to-proximal force transmission and torqueability to allow for advancement.

### Examples

Five different foam formulations are described below. All are intended for delivering a corticosteroid as the therapeutic agent but would be also suitable for at least most small molecule drugs.

### Examples 1 & 2

Two examples of foam formulations are presented in Table 1 below for dispensing in an air foam dispenser:

TABLE 1

| | |
|---|---|
| Example #1 | Example #2 |

| | | | | |
|---|---|---|---|---|
| Water | 50 | mL | 50 | mL |
| Tween ® 20 | 2 | mL | 2 | mL |
| Tween ® 80 | 2 | mL | 2 | mL |

| | | | |
|---|---|---|---|
| CMC | NA | 0.12 | g |

| | | | | |
|---|---|---|---|---|
| Glycerol | 5 | mL | 5 | mL |
| PEG400 | 5 | mL | 5 | mL |

| | | | |
|---|---|---|---|
| PEG1000 | NA | 0.2 | g |

| | | | | |
|---|---|---|---|---|
| API particles containing urea | 0.5 | g | 0.5 | g |

Add all components to water mixture while stirring at room temperature until homogenous. Dispense from an air foam dispenser.

### Example 3

Emulsification, ethanolic foam formulation containing therapeutic and excipient with pressurized foam dispenser.

TABLE 2

| Component | Component wt % |
|---|---|
| Ethanol | 58 |
| Cetyl alcohol | 1 |
| Stearyl alcohol | 0.5 |
| Tween ® 80 | 0.4 |
| Propylene glycol 2 | 2 |
| Water | 38 |
| API Therapeutic | 0.1 |

Stir together ethanol, cetyl alcohol, stearyl alcohol, Tween® 60, and propylene glycol at room temperature until homogenous. Combine two phase prior to foam formation. Dispense from a foam dispenser charged with N2O.

### Example 4

Aqueous formulation with particulate API suspension comprising nanoparticles having a size of ≤10 μm, and preferably within a range of 10 nm to 1 μm, and excipient. Aqueous formulation suitable for an air pump dispenser.

TABLE 3

| Component | Component wt % |
|---|---|
| Tween ® 20 | 2 |
| Water | 98 |
| API Therapeutic | 0.1 |

Dissolve Tween® in water. Dispense from a foam pump dispenser.

### Example 5

Emulsification, without ethanol, containing the therapeutic and excipient with an air pump dispenser.

TABLE 4

| Component | Component wt % |
|---|---|
| Propylene glycol | 11.5 |
| Tween ® 20 | 3 |
| HPC (Hydroxypropyl cellulose) | 0.1 |
| API Therapeutic | 0.1 |
| Water | qs add 100 |

Add foaming agent, stabilizing agent and therapeutic to oil phase. Add water. Agitate gently at 55° C. until dissolved. Cool to room temperature.

While the preferred embodiments of the devices and methods have been described in reference to the environment in which they were developed, they are merely illustrative of the principles of the present inventive concepts. Modification or combinations of the above-described assemblies, other embodiments, configurations, and methods for carrying out the invention, and variations of aspects of the invention that are obvious to those of skill in the art are intended to be within the scope of the claims. In addition, where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

What is claimed is:

1. A liquid drug delivery formulation for forming a foam when combined with a gas source, said formulation comprising:
water;
at least one hydrophilic solvent;
at least one surfactant or foaming agent; and
a drug particle comprising therapeutic agent combined with an excipient.

2. The liquid drug delivery formulation of claim 1, wherein the hydrophilic solvent is selected from the group consisting of alcohols and polyalcohols.

3. The liquid drug delivery formulation of claim 2, wherein the hydrophilic solvent is present at a concentration in a range from 5% to 75% by weight of the total liquid drug delivery formulation.

4. The liquid drug delivery formulation of claim 1, wherein the foaming agent or surfactant comprises an amphiphilic substance having hydrophilic and hydrophobic segments.

5. The liquid drug delivery formulation of claim 4, wherein the amphiphilic substance is selected from the group consisting of polysorbates, kolliphors, cetyl alcohol, stearyl alcohol, sorbitane monolaurate, polyoxyethylene stearate, polyoxamer, lecithin, laurates, oleates, and stearates.

6. The liquid drug delivery formulation of claim 4, wherein the amphiphilic substance is present at a concentration in a range from 0.25% to 5% by weight of the total liquid formulation.

7. The liquid drug delivery formulation of claim 6, wherein foaming agent or surfactant is present at a concentration at or near the critical micelle concentration for the foaming agent or surfactant.

8. The liquid drug delivery formulation of claim 1, wherein the therapeutic agent substance comprises a small molecule drug with a MW below 2000D.

9. The liquid drug delivery formulation of claim 8, wherein the therapeutic agent substance is formed as a nanoparticle.

10. The liquid drug delivery formulation of claim 9, wherein the nanoparticle has a size of ≤10 μm.

11. The liquid drug delivery formulation of claim 8, wherein the therapeutic agent is selected from the group consisting of beclomethasone dipropionate, budesonide, ciclesonide, flunisolide, fluticasone propionate, mometasone furoate, triamcinolone acetonide, azelastine, olopatadine, cetirizine, levocetirizine, desloratadine, fexofenadine, loratadine, montelukast, zileuton, diphenhydramine, hydroxyzine, chlorpheniramine, diphenhydramine, hydroxyzine, pseudoephedrine, cromolyn sodium, ipratropium, amoxicillin-clavulanate, cefpodoxime proxetil, cefuroxime, gatifloxacin, moxifloxacin, levofloxacin, oxymetazoline.

12. The liquid drug delivery formulation of claim 8, wherein the therapeutic agent is present at a concentration of <15% by weight of the total liquid formulation.

13. The liquid drug delivery formulation of claim 8, wherein the excipient is selected from the group consisting of urea, iopromide, citrate esters, and lipophilic antioxidants.

14. The liquid drug delivery formulation of claim 13, wherein the excipient is a lipophilic antioxidants selected from the group consisting of nordihydroguaiarectic acid, resveratrol, propyl gallate, butylated hydroxyl toluene, butylated hydroxyanisole, and ascorbate palitate.

15. The liquid drug delivery formulation of claim 1, wherein the weight ratio of drug-to-excipient in the nanoparticles is in a range from 3 to 1 to 0.5 to 1 by weight therapeutic agent-to-excipient.

16. The liquid drug delivery formulation of claim 1, further comprising one or more penetration enhancers to facilitate uptake to the tissue.

17. The liquid drug delivery formulation of claim 1, further comprising one or more hydrophobic solvents.

18. The liquid drug delivery formulation of claim 1, further comprising one or more stability enhancers.

19. The liquid drug delivery formulation of claim 1, further comprising one or more emulsifiers.

20. The liquid drug delivery formulation of claim 1, further comprising one or more solubility enhancers.

21. The liquid drug delivery formulation of claim 1, further comprising one or more thickening elements.

22. The liquid drug delivery formulation of claim 1, further comprising one or more gelling agents.

23. A method for generating a therapeutic foam, said method comprising:
incorporating a gas into the liquid drug delivery formulation of any one of the preceding claims.

24. The method of claim 23, wherein incorporating the gas in the liquid drug delivery formulation comprises pumping the liquid drug delivery formulation and the gas into a mixing chamber.

25. The method of claim 23, wherein incorporating a gas in the liquid drug delivery formulation comprises pressurizing a mixture of the liquid drug delivery formulation and the gas and passing the pressurized mixture through a valve to form an aerosol.

26. A method for treating a patient, said method comprising delivering foam generated as in claim 23 to a body cavity or lumen of the patient.

27. The method of claim 26, wherein the foam is delivered to a body cavity selected from the group consisting of nasal, buccal, ocular, gastro, vaginal, and rectal cavities.

28. The method of claim 27, wherein the foam is delivered to a nasal sinus.

29. The method of claim 28, wherein the sinus is at least one of a frontal sinus, a sphenoid sinus, an ethmoid sinus, and a maxillary sinus.

30. The method of claim 28, wherein a sufficient volume of foam is delivered to fill and occupy the nasal sinus.

31. The method of claim 28, wherein the foam is delivered through a catheter.

32. The method of claim 28, wherein the foam is concurrently with a stent.

33. The method of claim 28, further comprising washing or rinsing the nasal sinus prior to delivering the foam.

34. The liquid drug delivery formulation of claim 1, wherein the therapeutic agent substance is formed as a micelle.

35. The liquid drug delivery formulation of claim 1, further comprising one or more mucoadhesive agents.

36. The method of claim 26, wherein the at least one surfactant or foaming agent disrupts mucus and lipids on a wall of the body cavity or lumen to enhance delivery of the drug particles to a mucosal layer on the wall of the body cavity or lumen.

37. The method of claim 36, wherein the body cavity or lumen is a nasal sinus.
